(19) United States
(12) Patent Application Publication
(10) Pub. No.: US 2003/0169603 A1
(43) Pub. Date: Sep. 11, 2003

(54) Apparatus and method for illuminating a field of view within an eye

(76) Inventors: K. Peter Luloh, Longwood, FL (US); Michael Annen, Sanford, FL (US); Paul Tornambe, Poway, CA (US); Frank H.J. Koch, Frankfurt (DE)

Correspondence Address: BEUSSE, BROWNLEE, BOWDOIN & WOLTER, P. A., 390 NORTH ORANGE AVENUE, SUITE 2500, ORLANDO, FL, 32801, US

(21) Appl. No.: 10/376,814

(22) Filed: Feb. 28, 2003

Related U.S. Application Data
(60) Provisional application No. 60/361,846, filed on Mar. 5, 2002.

(51) Int. Cl. A61B 1/06

(52) U.S. Cl. 362/574; 362/572; 362/575; 362/554

(57) ABSTRACT

An apparatus and method for illuminating a region of an eye is provided and may include at least one optical fiber in fluid connection with a light source for allowing light to be transmitted to a probe for diffusing light into the region of the eye. The probe be formed from a portion of the at least one optical fiber and may be directly inserted into a portion of the eye so that light is diffused into the eye to illuminate the region. The probe may be secured in a substantially fixed position when the probe is inserted within a portion of the eye such as by a frictional engagement between the diffusing means and the eye. A plurality of probes may be inserted within the eye where each probe may include a substantially tapered distal end to facilitate insertion into the eye. The distal end of each probe may be formed in different shapes or may include an optical lens for diffusing light into the eye for illuminating the region of the eye during a surgical procedure.

### CROSS REFERENCE TO RELATED APPLICATIONS

This application claims priority to a provisional application filed on Mar. 5, 2002 having application Ser. No. 60/361,846, the specification of which is incorporated herein by reference.

### BACKGROUND OF THE INVENTION

This invention relates in general to eye surgery and more particularly to a method and system for internally lighting or illuminating an eyeball to enable a surgeon to perform various surgical procedures on the eye and allow the use of varying instruments during these procedures.

A common surgical procedure performed on eyes, for example, is pars plana vitrectomy. This procedure is a closed vitreous surgical technique for operating on the eye wherein the surgical field is observed through the pupil and instrumentation is inserted into the vitreous cavity through surgical cuts or sclerotomies. These cuts may be fitted with ports to prevent leakage of intraocular fluid during the procedure. Visualization is accomplished using a viewing system, such as a binocular indirect opthalmomicroscope system disclosed in U.S. Pat. No. 4,710,000 or 5,009,487. Intraocular pressure is regulated by infusion of fluid through a separate sclerotomy port. Illumination of the back of the eye or fundus may be originated from an external source through the pupil, or internally through fiber optics. It has been generally recognized that internal illumination with fiber optics is superior to external illumination and is not as dependent on variances in pupillary dilatation or clarity of the ocular media. A frequent practice is to employ a three- or four-port procedure, utilizing one or two ports for exchangeable working instruments, another port for infusion, and another port for illumination using a source such as a ceiling light available from D.O.R.C. Company, Geervleit, The Netherlands, or a chandelier system available from Grieshaber, Schafthausen, Switzerland.

It is known to incorporate optical fibers into the working end of the surgical instrument. This eliminates the need for a separate illumination port and offers the advantage of directing the light beam together with the instrument onto the target site. Instrument sizes must, however, be correspondingly increased and larger sclerotomies may be necessary. An alternative procedure is to employ an illuminated infusion cannula to integrate the infusion and illumination functions at a single point.

One example of a combined infusion cannula and illumination source is disclosed in U.S. Pat. No. 4,820,264. The '264 device comprises an infusion channel through which light transmitting fibers are passed for directing light into the eyeball at the point of discharge of the intraocular irrigating solution. Such illumination is not automatically directed by manipulation of the cutting instruments. Moreover, the fibers are run directly within the infusion channel, and illumination and infusion portions are non-separable near the eye. This may cause a less than optimal illumination of the area or field of view undergoing the surgical procedure.

The integrated lighting concept has been extended to provide illuminated cannulas at multiple ports having channels through which either infusion fluids or surgical instruments can be passed. Such a multiport illuminated cannula system is disclosed in U.S. Pat. No. 5,632,740. Such multiport illuminated cannula may comprise a plurality of light transmitting fibers annularly arranged about a central instrument-receiving working channel. Such device has the advantage that fibers are located external to the working channel. The channels may be, however, awkward to seal upon instrument removal and, if used for infusion purposes, may lack expedient infusion tube interfaces and, as in the '264 device, discharge fluid directly at the optical fiber terminations, thereby interfering with illumination.

A general problem with the above-described devices is that are complicated lighting systems and the channel for inserting instruments to the eye is inseparable from channels allowing for the illuminating sources to direct light to the surgical area or field of view. This combination may limit the surgical diversity of a device and/or create the need to move the device around during surgery to properly illuminate the surgical area or target. This need may result because the optical fibers directing light for illuminating the field of view concentrate the light too directly on a specific area or areas. Light directed in this manner may not be sufficiently diffused for continuously and uniformly illuminating a field of view. Furthermore, other illumination devices used during eye surgery, such as a common light pipe, may require that a surgeon hold or guide them in one hand during surgery. This type of mono-handed surgery may limit the range of a surgeon's procedures or impede the surgical precision necessary for performing eye surgery.

### SUMMARY OF THE INVENTION

In view of the above, it would be advantageous to provide an apparatus for illuminating a field of view during a procedure on an eye that delivers diffused light to the eye so that manipulation of the light source is not required during the procedure. This allows for a surgeon to perform the procedure without interruption for redirecting light and provides optimal illumination of the field of view. It would be further advantageous to provide such a light source as an independent instrument that does not need to be held in one hand during surgery once in position. This allows for a surgeon to use both hands during surgery to use and manipulate various surgical instruments without impairing the illumination of the field of view.

An apparatus for illuminating a region of an eye is provided that may include at least one optical fiber in fluid communication with a light source for providing light to a means for diffusing light within an eye. The means for diffusing light into the region of the eye may be connected with a distal end of the at least one optical fiber. A means for securing the diffusing means in a substantially fixed position when the diffusing means is inserted within a portion of the eye is also provided. In one exemplary embodiment, the diffusing means may be a substantially cylindrical probe made of a transparent or translucent material for allowing light to be admitted and diffused in the region of interest when inserted within the eye. The diffusing means may be secured in a substantially fixed position when inserted into the eye. In one exemplary embodiment the diffusing means may be held in place by a friction engagement established between an outer layer or surface of the diffusing means and an interior portion of the eye when the diffusing means is inserted into the eye.

One aspect allows for providing an apparatus for illuminating at least one field of view of an eye during surgery on the eye that may include a light source and means for transmitting light from the light source to a plurality of light emitting probes. At least one of the probes may be inserted within the eye such that an outer layer or surface of the at least one probe contacts an interior portion of the eye while inserted. One embodiment allows for a means for securing the probe to include a friction engagement established between the outer layer of a probe and a portion of an interior surface of the eye when the probe is inserted in the eye. Alternate securing means may include a hooked portion or bend in the transmitting means, such as an optical fiber, that is proximate a base of the at least one probe. The hooked portion may be configured in relation to the at least one probe such that a stress is created at an angle to a longitudinal axis of the at least one probe when inserted into the eye. In this respect, the stress created prevents the at least one probe from being unintentionally moved or pulled out of the eye and positions the optical fiber so it does not interfere with a surgeon while performing the surgery.

Another aspect allows for a method for illuminating a field of view within an eye, the method including inserting at least one light-diffusing probe directly into an eye such that the at least one probe extends into an interior portion of the eye and diffuses light into the eye to illuminate the field of view. The at least one probe may be inserted into the eye to a depth such that a distal end of the probe does not protrude into the field of view. The method may include securing the at least one probe in a substantially fixed position while inserted within the eye. A plurality of probes may be inserted proximate a peripheral edge of the eye for providing a uniform and continuous illumination of a field of view.

### BRIEF DESCRIPTION OF THE DRAWINGS

FIG. 1 is a plan view of an exemplary embodiment of the present invention;

FIG. 2 is an enlarged plan view of an exemplary embodiment of a distal end of an optical fiber shown in FIG. 1; and

FIG. 3 is an enlarged plan view of an exemplary embodiment of a distal end of an optical fiber shown in FIG. 1.

### DETAILED DESCRIPTION OF THE INVENTION

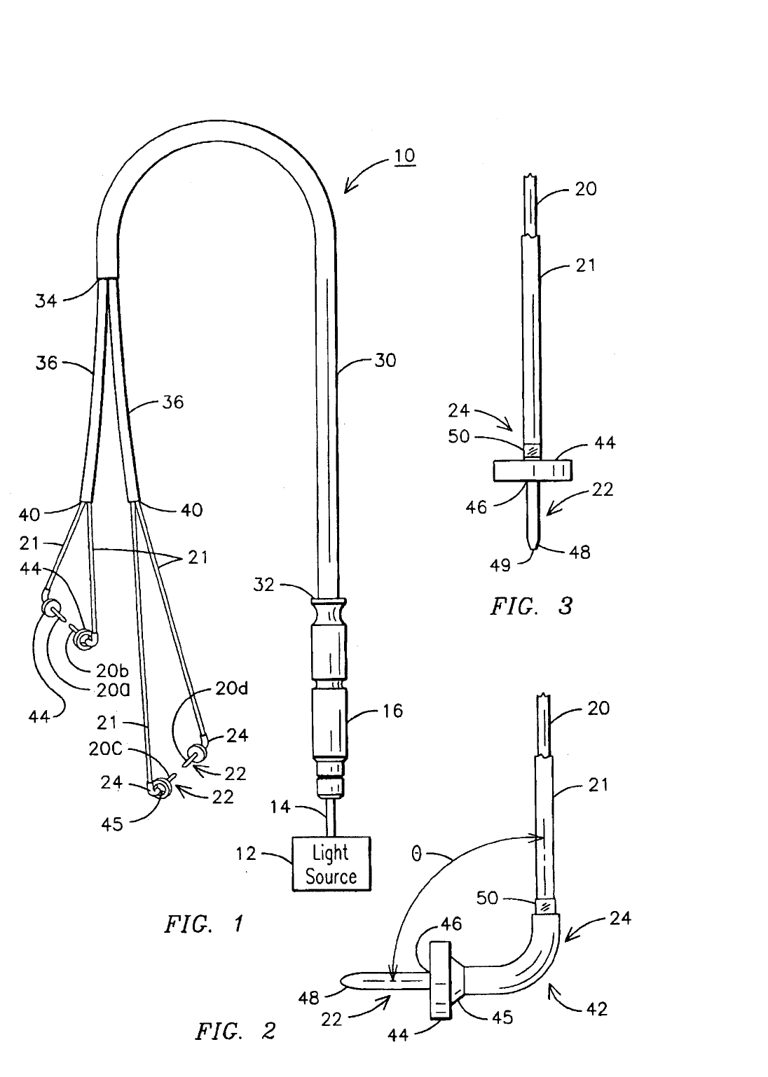

FIG. 1 illustrates an exemplary embodiment of an apparatus 10 in accordance with one aspect of the present invention. The apparatus 10 may receive light transmitted from a conventional light source 12 connected via a light-transmitting conduit 14 to a connector 16. The connector 16 may be constructed of aluminum for example and may be fitted with suitable connectors (not shown) for connecting a plurality of commercially available optical fibers 20, such as 25 gauge fibers, with the light source 12. In one exemplary embodiment, the length of each optical fiber 20 may be encased within an opaque outer layer 21 that may be a black polyvinyl chloride (“PVC”) such as a heat shrinkable PVC material, for example. Use of other suitable materials will be recognized by those skilled in the art. The outer layer 21 may be applied to prevent light from escaping the optical fibers 20 during transmission. Alternate embodiments allow for the optical fibers 20 to be used without application of the outer layer 21 as a function of operational or performance parameters of the apparatus 10 such as the light intensity requirements of a specific surgical procedure, for example. Other parameters may influence the properties of or need for outer layer 21 such as the optical fibers' 20 specifications and/or their length when connected to the light source 12.

The plurality of optical fibers 20 may function as a means for transmitting light to a plurality of light-emitting probes 22 disposed on a distal end 24 of the plurality of optical fibers 20. In one exemplary embodiment, each light-emitting probe 22 may be formed as a continuous portion of a respective one of the plurality of optical fibers 20. In this respect, the outer layer 21 may cover an optical fiber strand 20 so that a portion of the distal end 24 of the strand is exposed to function as the light-emitting probe 22. One embodiment allows for the optical fibers 20 to be selected having properties that will permit the length of probe 22 to function as a light diffusing means, such as a translucent optical fiber glass for example. An alternate embodiment allows for the optical fibers 20 to be selected having properties that will minimize light diffusion along the length of probe 22 so that the light is transmitted to the end of the probe 22 where it may be diffused into a field of view within the eye as more fully described below. A sheath or tubing 30 may be provided and encase the plurality of optical fibers 20 to protect them from damage, contain light within the fibers and maintain them in a bundle for ease of handling and connection to the connector 16. The tubing 30 may be fabricated of conventional flexible material such as black silicon tubing, for example. The tubing 30 may extend from an upper end 32 of the connector 16 to a dividing point 34 where the plurality of optical fibers 20 may split into discrete groups. One exemplary embodiment of the apparatus 10 allows for four optical fibers 20 to be bundled within the tubing 30 then split into two groups of two at the dividing point 34 where each group may be encased by a sheath or tubing 36. The tubing 36 may be fabricated of conventional flexible material such as black PVC tubing, for example. The tubing 36 may extend from the dividing point 34 to an exiting point 40 where the optical fibers 20 may no longer be encased by tubing 36. This allows for each of the optical fibers 20 to be manipulated as a single optical fiber strand for ease of a surgeon's placement within an eye. Each optical fiber 20 may emerge from the tubing 36 at exit point 40 with or without the opaque outer layer 21 as a function of surgical specifications, for example. The tubing 30 and 36 provide protection to the optical fibers 20 and help to contain light within the fibers. They also provide a convenient way to control the bundled fibers and place the light-emitting-probes 22 for a surgical procedure. Bundling a portion of the fibers also helps to ensure that the fibers 20 do not interfere with a surgeon or technician during surgery. For example, the light source 12 may be located several feet away from the point at which the probes 22 are being used during surgery. Tubing 30 and 36 allow for the single fiber optic strands 20 to be bundled together up to the point where a surgeon needs each strand to be flexible and individually manipulated. Alternate embodiments allow for tubing 30 to extend to the exiting point 40 in which case each of the plurality of optical fibers 20 would exit tubing 30 as individual strands. Another alternate embodiment allows for each optical fiber 20 to exit from the connector 16 as an individual strand. Other configurations for bundling or controlling the plurality of optical fibers 20 will be apparent to those skilled in the art.

FIG. 2 shows an enlarged illustration of a distal end 24 of an optical fiber 20 of FIG. 1. One embodiment of apparatus 10 allows for a bend or elbow 42 to be formed in the distal end 24 of one or more of the optical fibers 20. Bend 42 may be formed by encasing an optical fiber 20 within outer layer 21 where the outer layer 21 is composed of a material having an appropriate stiffness in the bending area to maintain the bend 42 in substantially the same shape over time. Bend 42 may also be formed with an appropriate flex so that it may be adjusted depending on the specific application. The bend 42 may cause an angle θ of approximately 90 degrees to be formed between a longitudinal axis of the probe 22 and a longitudinal axis of a corresponding optical fiber 20. The bend 42 may function as a means for securing a probe 22 in a substantially fixed position when inserted into an eye. This may be accomplished by causing a stress or force to be created or exerted at an angle of approximately 90 degrees, which is approximately equivalent to angle θ, to the longitudinal axis of the probe 22 when inserted into the eye. This stress or force will restrict the movement of the probe 22 once in place so that a continuous and uniform dispersion of light may illuminate a field of view desired by a surgeon during surgery or other procedure. Alternate embodiments allow for angle θ to be of varying degrees in response to varying applications of apparatus 10. For example, the angle to horizontal with which a light-emitting probe 22 is inserted into an eye during a procedure may be such that the angle θ may need to be acute or obtuse, for example, to create a sufficient stress or force to secure the probe 22 in a substantially fixed position during the procedure. Those skilled in the art will recognize that other factors such as the dimensions or shape of probe 22 and/or the depth to which it is inserted into an eye, for example, may also influence the size of angle θ.

A means for stopping a probe 22 from being inserted into a portion of the eye beyond a predetermined or desired distance, such as collar or footplate 44, may be provided in an exemplary embodiment of the present invention. The footplate 44 may be constructed of a suitable material such as a surgical grade silicone. In one embodiment, the footplate 44 may by substantially circular having a diameter of approximately ⅛ of an inch but may vary depending on the application. A surface of the footplate 44 facing a probe 22 may abut an exterior portion or surface of the eye when probe 22 is inserted a predetermined or desired depth into the eye. An optical fiber 20 may be inserted through an aperture (not shown) in the footplate 44 to move it into position. One advantage of using the surgical silicone is that it has self-sealing properties so that a tight, rigid seal may be formed around a base portion 46 of the probe 22. It will be recognized by those skilled in the art that other materials may be used having varying shapes or sizes to perform as a means for stopping a probe 22 from being inserted too far into an eye. An alternate embodiment allows for a probe 22 to be inserted without a stopping means 44. In this respect, placement of the probe 22 could be determined by its length, bend 42 and/or a guide mark on its surface, for example. A tapered portion 45 may extend from the footplate 44 that provides a surface area for affixing the stopping means or footplate 44 to the bend or elbow 42. For example, an epoxy or other appropriate means for affixing footplate 44 to elbow 42 may be applied to their respective exterior surfaces to bond them together. Alternate means for affixing footplate 44 to elbow 42 will be recognized by those skilled in the art. Another exemplary embodiment shown in FIG. 3 allows for the footplate 44 to abut the outer layer 21 of fiber optic strand 20 with the footplate 44 being held in place by an interference or friction fit, for example. The tapered portion 45 could also be adapted for use with the embodiment of FIG. 3.

As shown in FIG. 2, the light-emitting probe 22 may be fabricated so a portion functions as a means for diffusing light into the eye, such as distal end 48, both of which may be a polished fiber optic glass. In this respect, distal end 48 may be symmetrically tapered, conical, parabolic, spherical, cut at an angle, bullet shape or other configurations, for example, to achieve light diffusion properties commensurate with surgical specifications. In addition to diffusing light to obtain a wide field of illumination, a symmetrically tapered distal end 48, for example, facilitates insertion of the probe 22 into a region of the eye, which may be initiated through an incision. In one exemplary embodiment, probe 22 may be substantially cylindrical and sized so the distal end 48 does not physically protrude into a field of view. The probe 22 may be sterilized, lubricated or otherwise treated with known antibacterial material prior to insertion. Cylindrical probe 22 and distal end 48 may be part of an optical fiber 20 and be fabricated of a fiber optic glass or plastic such that the distal end 48 and/or the cylindrical portion of probe 22 act as a lens or means for diffusing light into an eye. Such fiber optic glass or plastic allow for light to be emitted into the eye in a diffused manner rather than being concentrated at a particular point or points. The diffusion of light allows for a continuous, uniform and wide field of illumination of at least one field of view needed by a surgeon for performing surgery or other procedures. Alternate materials for diffusing light may be used as will be recognized by those skilled in the art. Further, aspects of the present invention allow for light-emitting probe 22 and distal end 48 to be constructed to minimize light diffusing and maintain a more focused beam of light within a field of view as a function of the specific surgical procedure being performed. A portion of probe 22 may be covered with an outer layer 21 to achieve specific light diffusion effects.

FIG. 3 illustrates another exemplary embodiment of a distal end 24 of a corresponding optical fiber or strand 20 in accordance with one aspect of the present invention. This exemplary embodiment allows for the optical fiber 20 and the probe 22 to be formed along the same longitudinal axis. The distal end 24 may include a frusto-conical portion 48 having a means for diffusing light such as lens 49 secured thereto. The lens 49 may be formed of commercial optical glass, for example, and may be formed of various shapes and sizes as a function of the light diffusion specifications for a surgical procedure. An alternate exemplary embodiment allows for the frusto-conical section 48, which in one aspect may function as a means for diffusing light, to be eliminated in which case the lens 49 may have a diameter substantially the same as the optical fiber 20. Another alternate embodiment allows for the distal end 48 to be cut at an angle or slant so that the lens 49 may be held obliquely relative to the probe's 22 longitudinal axis. When an embodiment such as that of FIG. 3 is used for illumination of the eye, the means for securing the light-emitting probe 22 within the eye may be a frictional engagement between an outer surface of the probe 22 and an interior surface of the eye when the probe 22 is inserted in the eye and/or the frictional engagement may be established proximate an incision in the eye. An alternate embodiment allows for the means for securing to be conventional screw-in type receptacles that may be inserted into and held in place within incisions made in the eyeball, as with other embodiments of the invention. The probes 22 may pass through respective receptacles and into the eyeball.

FIG. 1 illustrates that in one embodiment the optical fibers 20, shown covered with an opaque layer 21, may have different lengths as a function of surgical needs. For example, one exemplary embodiment allows for two of the optical fibers 20a and 20b to constitute a first pair of optical fibers having a first length that is shorter than the a second length of a second pair of optical fibers 20c and 20d. This embodiment allows for the light-emitting probes 22 of respective optical fibers 20a and 20b to be inserted into a near side of an eye and the probes 22 of respective optical fibers 20c and 20d to be inserted in a far side of the eye relative to the direction of origin of tubing 30, for example. Those skilled in the art will recognize that optical fibers 21 may vary in their lengths and quantity as a function of surgeon, surgical, logistical, equipment, power supply and/or operating room needs, for example. One exemplary embodiment of the apparatus 10 allows for four light-emitting probes 22 to be inserted into an eyeball for internally lighting the eye or illuminating a field of view for surgery such as the back of the eye, for example. Each probe 22 may be inserted into the eye at different locations to provide a continuous and uniform diffusion of light into the eye to obtain a wide field of illumination during surgery or the conducting of other procedures. One aspect allows for each optical fiber 20a and 20b, for example, to include a means for identifying its location such as a reflective marker 50 as more clearly shown in FIG. 3. The reflective marker 50 may be affixed to the outer layer 21, the optical fiber 20 and/or the footplate 44, for example. Optical fibers 20a and 20b may each be provided with a marker 50 to indicate to a surgeon which pair of the fiber optic strands 20 has the shorter length. This is advantageous to the surgeon in a dimly lit operating room for determining which optical fibers to insert in locations of the eye when preparing for surgery. Alternate embodiments allow for markers 50 to be placed on fibers 20c and 20d in lieu of 20a and 20b or a color-coded system could be adopted for indicating to a surgeon which fibers are which. Markers 50 may also be used by a surgeon to determine the position of a respective optical fiber 20 before, during and after a surgical procedure.

One aspect of the present invention allows for a method of manufacturing the apparatus 10 that may include the step of selecting at least one optical fiber 20, which may be construction of glass or plastic for example, to be in fluid communication with a light source 12 for transmitting light to an eye for illuminating a field of view during surgery or other procedures. If the optical fiber 20 includes an outer layer, such as opaque outer layer 21, the step of stripping a portion of the outer layer to expose a length of optical fiber 20 may be performed. An alternate embodiment allows for an optical fiber 20 having no opaque layer to be covered with an opaque material to form the layer such as a heat shrinkable PVC material, for example. A portion of the exposed optical fiber glass may then be heated and a bend or elbow 42 may be formed in the optical fiber. One aspect allows for the glass to cool in ambient temperature or alternatively the glass may be exposed to a cooling chamber for accelerated cooling. The at least one optical fiber 20 may be selected to include four individual optical fibers. A stiff PVC elbow 42 may be placed on a flexible optical fiber 20 made of plastic to form a bend in the fiber. The four optical fibers 20 may include two pairs with each pair having different lengths. A reflective marker 50 may be applied to one or more of the optical fibers 20 and in one aspect a reflective marker 50 may be applied to each optical fiber 20 of the pair of fibers having a length that is shorter than the other pair of fibers. An elbow 42 fabricated of PVC, for example, may be applied over the bend formed in the distal end 42 of the at least one optical fibers 20. A stopper or footplate 44 may then be applied on the distal end 42 of the at least one optical fibers 20. In one exemplary embodiment a lens 49 may be integrated within the distal end 42 of the at least one optical fiber 20 for diffusing light to obtain a wide field of illumination in a field of view within an eye. Alternate exemplary embodiments allow for the light diffusing means to be formed in the distal end 42 of the at least one optical fiber 20 such as a symmetrically tapered, conical, parabolic or bullet shape 48, for example, as a function of performance specifications.

While exemplary embodiments in accordance with aspects of the present invention have been shown and described by way of example only, numerous variations, changes and substitutions will occur to those of skill in the art without departing from the invention herein. Accordingly, it is intended that the invention be limited only by the spirit and scope of the appended claims.

We claim

1. An apparatus for illuminating a region of an eye, the apparatus comprising:
at least one optical fiber in fluid connection with a light source;
means for diffusing light in fluid connection with the at least one optical fiber and adapted to be inserted into an eye for illuminating the region; and
means for securing the diffusing means in a substantially fixed position when inserted within a portion of the eye.

2. The apparatus of claim 1 wherein:
the diffusing means is insertable within an incision made in the eye such that an outer surface of the diffusing means directly contacts an interior portion of the eye.

3. The apparatus of claim 2, the securing means comprising a friction engagement established between the outer surface of the diffusing means and the interior portion of the eye.

4. The apparatus of claim 2, the diffusing means comprising a substantially cylindrical probe having a substantially tapered distal end.

5. The apparatus of claim 1, the securing means comprising a friction engagement between an outer surface of the diffusing means and at least an interior portion of the eye when the diffusing means is inserted into the eye.

6. The apparatus of claim 1, the diffusing means comprising a substantially cylindrical probe and an optical lens disposed within a distal end of the probe.

7. The apparatus of claim 1, the securing means comprising a hooked portion proximate the distal end of the optical fiber, the hooked portion configured in relation to the diffusing means such that a stress is created at an angle to a longitudinal axis of the diffusing means when the diffusing means is inserted into the eye.

8. The apparatus of claim 7, the hooked portion comprising a bend formed in the at least one optical fiber.

9. The apparatus of claim 1 further comprising:
means for stopping the diffusing means from being inserted into the portion of the eye beyond a predetermined distance.

10. The apparatus of claim 9, the means for stopping comprising a substantially circular footplate proximate a base of the diffusing means.

11. The apparatus of claim 1 further comprising:
an opaque outer layer covering a portion of the optical fiber.

12. The apparatus of claim 1 wherein the diffusing means is formed as part of a distal end of the at least one optical fiber.

13. The apparatus of claim 1 further comprising:
a reflective marker proximate the diffusing means.

14. An apparatus for illuminating a field of view of an eye during a procedure being performed on the eye, the apparatus comprising:
a light source; and
means for transmitting light from the light source to a plurality of light-diffusing probes where at least one of the probes is insertable within the eye such that an outer surface of the at least one probe contacts an interior portion of the eye while inserted.

15. The apparatus of claim 14 further comprising:
means for securing the at least one probe in a substantially fixed position outside the field of view when inserted into the eye.

16. The apparatus of claim 15, the securing means comprising a friction engagement between the outer layer of the at least one probe and the interior portion of the eye.

17. The apparatus of claim 15, the securing means comprising a hooked portion proximate a base of the at least one probe, the hooked portion configured in relation to the at least one probe such that a stress is created at an angle to a longitudinal axis of the at least one probe when inserted into the eye.

18. The apparatus of claim 14 further comprising:
means for stopping the at least one probe from being inserted into the eye beyond a predetermined distance.

19. The apparatus of claim 18, the means for stopping comprising a footplate proximate a base of the at least one probe.

20. The apparatus of claim 14, the transmitting means comprising a plurality of optical fibers.

21. The apparatus of claim 20, the plurality of optical fibers comprising:
a first pair of optical fibers having a first length; and
a second pair of optical fibers having a second length wherein the first length is longer than the second length such that a first pair of probes attached to a respective one of the first pair of optical fibers may be inserted within a first side of an eye and a second pair of probes attached to a respective one of the second pair of optical fibers may be inserted within a second side of the eye.

22. The apparatus of claim 21 further comprising:
a reflective marker attached to at least one optical fiber.

23. The apparatus of claim 21 further comprising:
a reflective marker attached to each optical fiber of the second pair of optical fibers.

24. The apparatus of claim 14, the means for transmitting comprising:
a plurality of optical fibers wherein each of the probes comprises a portion of a distal end of a respective optical fiber.

25. The apparatus of claim 24 further comprising:
an optical lens disposed with the portion of the distal end.

26. The apparatus of claim 24 wherein the portion of the distal end is substantially tapered.

27. A method for illuminating a field of view within an eye, the method comprising:
inserting at least one light diffusing probe in fluid communication with a light source directly into an eye such that the at least one probe extends into an interior portion of the eye and diffuses light into the eye to illuminate the field of view.

28. The method of claim 27 wherein the at least one probe is inserted into the eye to a depth such that a distal end of the probe does not protrude into the field of view.

29. The method of claim 27 further comprising:
securing the at least one probe in a substantially fixed position while inserted within the eye.

30. The method of claim 29 wherein the at least one probe is secured in a substantially fixed position by a friction engagement between an outer surface of the probe and a region of the eye within which the probe is inserted.

31. The method of claim 29 wherein the at least one probe is secured in a substantially fixed position by a hooked portion proximate a base of the at least one probe, the hooked portion configured in relation to the at least one probe such that a stress is created at an angle to a longitudinal axis of the at least one probe when inserted into the eye.

32. The method of claim 27 further comprising:
determining a plurality of insertion points for inserting a respective one of the at least one probes into the eye wherein the plurality of insertion points are disposed proximate a peripheral edge of the eye and wherein a respective probe is inserted into the eye proximate a respective insertion point.

33. The method of claim 32 further comprising:
making an incision proximate at least one of the insertion points.

34. The method of claim 27 wherein the at least one probe includes a substantially tapered distal end.

35. The method of claim 27, the step of inserting further comprising:
inserting a first pair of the light-diffusing probes within a first side of the eye, the first pair of probes connected with the light source by a first pair of optical fibers having a first length; and
inserting a second pair of the light-diffusing probes within a second side of the eye, the second pair of probes connected with the light source by a second pair of optical fibers having a second length wherein the first length is longer than the second length.
